(19) United States
(12) Patent Application Publication
(10) Pub. No.: US 2022/0301677 A1
(43) Pub. Date: Sep. 22, 2022

(54) PLATFORM AND INTERFACES FOR MANAGING CAREGIVERS

(71) Applicant: RSV Opco 5, Inc. d/b/a Kinto, Cambridge, MA (US)

(72) Inventors: Joseph Taisup Chung, Cambridge, MA (US); Emily Jayne Silver, Portland, ME (US); Samuel Kenton Davies, Shutesbury, MA (US); Brett Maden, Boston, MA (US); Richard Curtis, Marblehead, MA (US); Duncan Macdonald Pettigrew, Maden, MA (US)

(21) Appl. No.: 17/699,276

(22) Filed: Mar. 21, 2022

Related U.S. Application Data
(60) Provisional application No. 63/164,178, filed on Mar. 22, 2021.

(51) Int. Cl. G16H 20/10; G06F 3/0482

(52) U.S. Cl. CPC G16H 20/10; G06F 3/0482

(57) ABSTRACT

Provided is a dynamic, customizable, and manage user interface that supports a coaching environment of a mobile application for caregivers. The dynamic user interface links respective applications of caregivers to care coaches, and dramatically changes the care environment, and enables full utilization of the care coaching support, tools, and information. A new technical architecture is provided that is configured to support caregivers by emulating the caregiver's device/applications executing on a caregiver's device to a respective coaching device monitored by a care coach. Through the dynamic user interface coaches can introduce new functionality into the emulation and trigger the introduction and/or execution of the new functionality on a targeted caregiver device via the care coach interface. The unique architecture enables dynamic changes to the features, displays, and/or functions available on caregiver systems, that occur in real time or based on communicating changes in a respective display/emulation.

### RELATED APPLICATIONS

This application is a Non-Provisional of and claims priority to Provisional (35 USC 119(e)) of U.S. Application Ser. No. 63/164,178, filed Mar. 22, 2021, entitled “PLATFORM AND INTERFACES FOR MANAGING CAREGIVERS”, which application is incorporated herein in its entirety.

### BACKGROUND

Many conventional systems exist that provide online advice and/or coaching to improve the experience for a care recipient and improve the level of care provided. Fewer systems are available to direct and/or coach caregivers in the task of providing care to a care recipient.

### SUMMARY

The inventors have realized that there is a need for an interactive tool that can facilitate the role of the caregiver. For example, a child who is caring for elderly parents needs far more support than provided by current solutions. According to one aspect, the inventors have realized that delivery of support information to caregivers alone is insufficient, and results in the delivered information being unutilized and/or the caregiver being overwhelmed by the process of caring for a loved one, among other issues.

In further aspects, the inventors have realized that a coaching environment supported by mobile applications for caregivers linked to applications for care coaches dramatically changes the care environment, and enables full utilization of the care coaching support, tools, and information. According to some embodiments, a new technical architecture is provided that is configured to support caregivers by emulating the caregiver's device/applications executing on a caregiver's device to a respective coaching device monitored by a care coach. This approach is not available, nor is the functionality available in many conventional approaches and/or implementations. Moreover, care coaches can introduce new functionality into the emulation and trigger the introduction and/or execution of the new functionality on a targeted caregiver device via the care coach interface. The linked architecture enables introduction of new functionality to a caregiver device and enables functionality unavailable in conventional systems. Further, the unique linked architecture enables dynamic changes to the features, displays, and/or functions available on caregiver applications and/or devices, that can occur in real time or based on propagating changes in a respective display/emulation.

According to one embodiment, care coaches are linked to one or more caregivers that form a care group managed by the one or more care coaches. In various embodiments, care coaches are given access to visualize their caregivers' applications and interfaces. For example, the application interface provided to each care coach enables unique and fully integrated functionality to assist their linked caregivers. In one example, the coach interface enables the care coach to interact with a mobile application used by their caregiver in real time. In another example, the coach can trigger new elements to appear in the caregiver's UI. Further, the coach can do so in the context of answering questions, providing support, and/or teaching the caregiver what options or choices are available, which can include what additional support options are available to the caregiver (e.g., external systems, care group support, additional information material, etc.). In various embodiments, a care coach view can be configured for real time display of respective caregiver devices, and can be configured to emulate a known state of the caregiver device (e.g., tracked via profile information). Some embodiments of the system enable the care coach to interact with the caregiver device both in real time and as an emulation that triggers updates to the caregiver device. The caregiver's view is also updated based on changes made on the caregiver device.

According to one aspect, a system for enabling a customizable and managed user interface is provided. The system comprises a remote computer subsystem including at least one processor operatively connected to a memory, the at least one processor configured to access, via a web interface, a central computer system, the central computer system for managing at least one caregiver device and at least one remote computer subsystem associated with a care coach, access state information for an application installed on a caregiver device or access an emulation of the application executing on the caregiver device, generate a coach console interface for the care coach on the remote computer subsystem, wherein the generation of the coach console interface includes operations to display an application view emulating the application executing on the caregiver device, display a library of visual capsules, wherein at least some of the visual capsules include information on completing a care giving task and information requests to define a condition of a care recipient, and are configured to guide the caregiver through a plurality of displays to convey or capture associated information, wherein the visual capsules are associated with a respective display element, and responsive to selection of at least one visual capsule in the coach console interface, trigger updates to the user interface for the application executing on the caregiver device to include display of the at least one visual capsule's associated display element.

According to one embodiment, the at least one processor is configured to update the application view emulating the application executing on the caregiver device to include the selected at least one visual capsule upon execution of the at least one visual capsule executing on the caregiver device. According to one embodiment, the system further comprises the central computer system, and wherein the central computer system is configured to maintain state information on respective caregiver devices, wherein the state information includes application execution information including at least a list of visual capsules installed. According to one embodiment, the central computer system is configured to update the state information associated with respective caregiver devices responsive to changes made in the application view. According to one embodiment, the central computer system is configured to communicate updates to respective caregiver devices and respective applications based on the updates to the state information.

According to one embodiment, the central computer communicates updates to respective caregiver devices in real or near real time. According to one embodiment, the central computer communicates updates to respective caregiver devices and the application view of the respective caregiver devices in real or near real time. According to one embodiment, the coach console interface includes the application view and an organization of the library of the visual capsules into sections of a user interface. According to one embodiment, the coach console interface includes the application view and an organization of the library of the visual capsules into sections of a user interface including a complete section, an assigned section, and an unassigned section. According to one embodiment, the application view is further configured to emulate operations performed by the application executing on the caregiver device. According to one embodiment, operations performed within the application view are mirrored within the application executing on the caregiver device.

According to one aspect, a method for managing a customizable user interface is provided. The method comprises accessing, at least one processor, a central computer interface for managing at least one caregiver device and at least one management subsystem associated with a care coach, accessing, by the at least one processor, state information for an application installed on a caregiver device or access an emulation of the application executing on the caregiver device, the state information and emulation reflecting application status and user interface settings for the application, generating, by the at least one processor, a coach console interface for the care coach on the at least one management subsystem, wherein the act of generating includes, displaying, by the at least one processor, an application view emulating the application executing on the caregiver device, displaying, by the at least one processor, a library of visual capsules, wherein at least some of the visual capsules include information on completing a care giving task and information requests to define a condition of a care recipient, and are configured to guide the caregiver through a plurality of displays to convey or capture associated information, and wherein the visual capsules are associated with a respective display element, and responsive to selection of at least one visual capsule in the coach console interface, triggering, by the at least one processor, updates to the user interface for the application executing on the caregiver device to include display of the at least one visual capsule and the associated display element.

According to one embodiment, the method further comprises updating the application view emulating the application executing on the caregiver device to include the selected at least one visual capsule upon execution of the at least one visual capsule executing on the caregiver device. According to one embodiment, the method further comprises maintaining, by a central computer system, state information on respective caregiver devices, wherein the state information includes application execution information including at least a list of visual capsules and display organization. According to one embodiment, the method further comprises updating the state information associated with respective caregiver devices responsive to changes made in the application view. According to one embodiment, the method further comprises communicating updates to respective caregiver devices and respective applications based on the updates to the state information.

According to one embodiment, the method further comprises communicating updates to respective caregiver devices in real or near real time. According to one embodiment, the method further comprises communicating updates to respective caregiver devices and the application view of the respective caregiver devices in real or near real time. According to one embodiment, the coach console interface includes the application view and an organization of the library of the visual capsules into sections of a user interface including a complete section, an assigned section, and an unassigned section. According to one embodiment, the method further comprises displaying in a coach console interface the application view and a display of the library of the visual capsules. According to one embodiment, the method further comprises displaying the application view to emulate operations performed by the application executing on the caregiver device. According to one embodiment, the method further comprises mirroring operations performed within the application view within the application executing on the caregiver device.

Still other aspects, embodiments, and advantages of these exemplary aspects and embodiments, are discussed in detail below. Any embodiment disclosed herein may be combined with any other embodiment in any manner consistent with at least one of the objects, aims, and needs disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. The appearances of such terms herein are not necessarily all referring to the same embodiment. The accompanying drawings are included to provide illustration and a further understanding of the various aspects and embodiments, and are incorporated in and constitute a part of this specification. The drawings, together with the remainder of the specification, serve to explain principles and operations of the described and claimed aspects and embodiments.

### BRIEF DESCRIPTION OF THE FIGURES

Various aspects of at least one embodiment are discussed herein with reference to the accompanying figures, which are not intended to be drawn to scale. The figures are included to provide illustration and a further understanding of the various aspects and embodiments, and are incorporated in and constitute a part of this specification, but are not intended as a definition of the limits of the invention. Where technical features in the figures, detailed description or any claim are followed by reference signs, the reference signs have been included for the sole purpose of increasing the intelligibility of the figures, detailed description, and/or claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. In the figures:

FIG. 1 shows example screen captures and caregiver functionality, according to one embodiment;

FIG. 2 shows example screen captures and caregiver functionality, according to one embodiment;

FIG. 3 is an example screen capture for a care coach console, according to one embodiment;

FIG. 4 is an example screen capture, according to one embodiment;

FIG. 5 is an example screen capture, according to one embodiment;

FIG. 6 is an example screen capture, according to one embodiment;

FIG. 7 is an example screen capture, according to one embodiment;

FIG. 8 is an example screen capture, according to one embodiment;

FIG. 9 is a block diagram of an example computer system that is improved based on specially configuring the computer system to execute the functions described herein, according to one embodiment;

FIG. 10 is a block diagram of an example system, according to one embodiment; and

FIG. 11 is an example process flow for updating a caregiver application, according to one embodiment.

### DETAILED DESCRIPTION

According to various aspects, a care system is configured to provide a technology-implemented care coaching platform for caregivers. Various embodiments have been implemented and tailored to support caregivers of patients with Alzheimer's Disease and related dementia, and/or cognitive challenges, however, further embodiments of the platform are configured to support the mobile application and linked care coach to help caregivers of patients with any condition. In one example, the care system and/or platform includes a mobile application (e.g., named Kinto), which leverages ubiquitous mobile technology to enable a more efficient coaching interface relative to known approaches. Care coaches can utilize a unique interface to interact directly with a caregiver view of the caregiver's mobile application. The care coach interface provides improved operation, limiting the computation burden (e.g., only integrating functions and features needed as needed) relative to conventional approaches, and limiting storage/bandwidth required for caregivers or caregiver groups to access information relevant to their needs, and/or improving a care coach's ability to interact with multiple care recipients at any given time.

These technological improvements yield a system that outperforms and improves over various conventional implementations. For example, care coaches utilizing the system can handle far greater caseloads, and caregiver interaction, than various existing solutions, including, for example, telephone-based or in-person implementation. Various embodiments are configured to leverage an innovative embedded user interface that is displayed to care coaches accessing the system. For example, the care coach can interact with an embedded “App View” of the Kinto Mobile app installed at caregiver devices. According to one embodiment, the App View can be integrated with a web-based coach console that can be accessed on respective care coach computer systems/devices. In various embodiments, the system leverages the integrated App View to enable a care coach to visualize the same screens that their caregiver is using/seeing. In this environment, the care coach can efficiently guide the caregiver and even perform actions on their behalf that are reflected on their mobile application display. In various embodiments, the care coaches are able to more efficiently utilize available functions, filter system features to relevant selections, and deliver a more efficient experience to the caregiver, among other options.

In some alternatives and/or additional embodiments, the care system is architected with a flexible data model and flexible interface that displays custom actions and/or activities under a “homework” model. In various embodiments, various content and/or functionality is presented as a capsule (e.g., user interface display element or visual capsule), and in further example, homework capsules, that a user can select to expand and or trigger operation. Each capsule can contain relevant educational information, information capture screens, actions and/or activities to perform, connections to additional support services, advice on caregiving activity, preparation steps for engaging in caregiving or care providing by a care service, among other options. In various embodiment, the system enables updates to and introduction of new functionality in various mobile applications based on updating or introducing various visual capsules into the application. The visual capsule are user interface elements that operate as containers for information, functionality, and/or operations that are executed by the mobile application.

In some embodiments, the system is configured to emulate the mobile application installed at a caregiver devices and any respective visual capsules shown in the respective user interface. For example, the system can include a profile of a current application view, settings, etc., of a respective caregiver device that a care coach can access and also update. In some examples, the care coach can update the emulation of the mobile application to introduce new functions, new display elements, update existing displays, re-organize features, etc. The changes to the mobile application emulation can be stored (e.g., in a device or user profile), and pushed to an executing mobile device when activated or connected to the system. In some embodiments, the care coach can introduce new visual capsules that can include a collection of any combination of information, display screens, functions, operations, etc., executable within the mobile application.

Examples of the methods, devices, and systems discussed herein are not limited in application to the details of construction and the arrangement of components set forth in the following description or illustrated in the accompanying drawings. The methods and systems are capable of implementation in other embodiments and of being practiced or of being carried out in various ways. Examples of specific implementations are provided herein for illustrative purposes only and are not intended to be limiting. In particular, acts, components, elements, and features discussed in connection with any one or more examples are not intended to be excluded from a similar role in any other examples.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element, or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

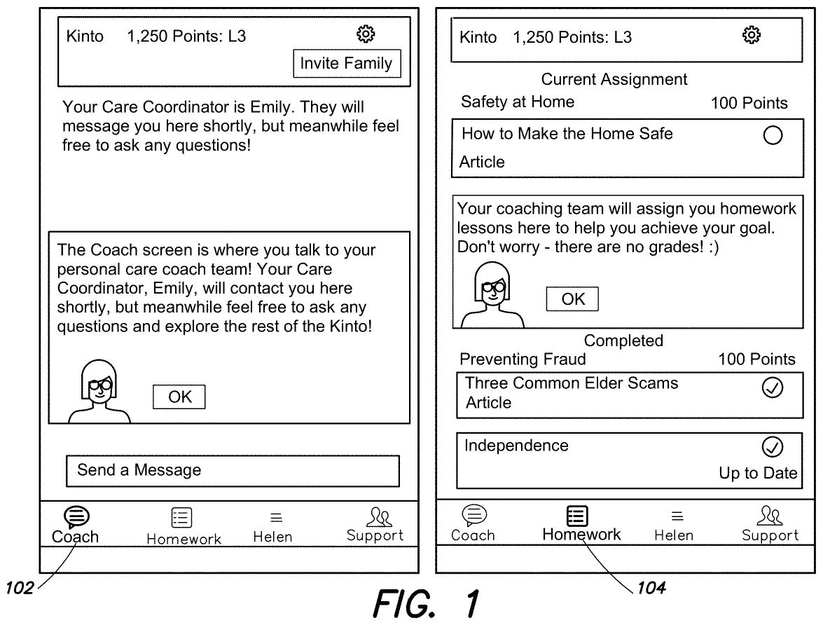

FIG. 1 illustrates example screen captures of a mobile device user interface that caregivers can access as part of the system. In FIG. 1, the navigation bar appearing at the lower portion of the screen includes navigation selections to access caregiver functionality and/or interact with a care coach. For example, at 102 the user can access coach functions on their mobile application. In some embodiments, the coach screen can be the initial display by default. The homework display at 104 transitions the user to the example display shown on the right side of FIG. 1.

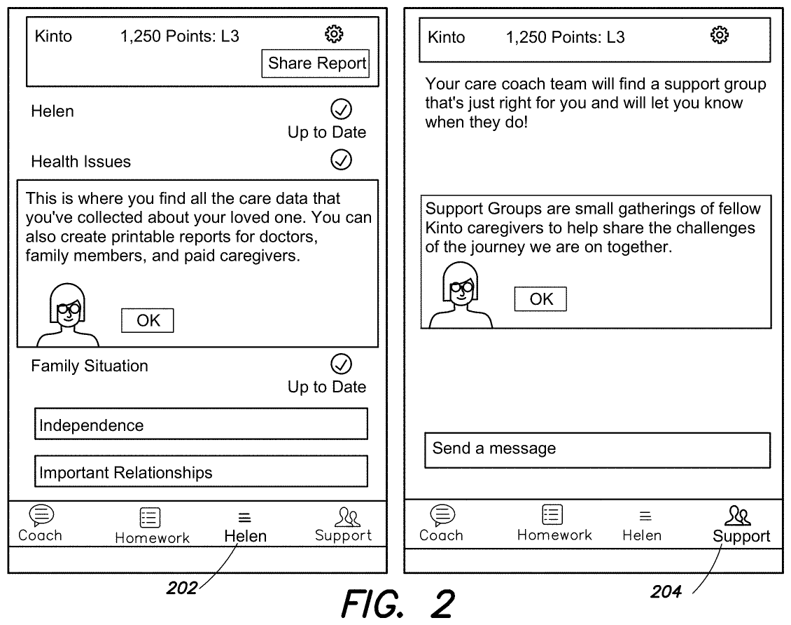

Homework capsules are digital objects of information, actions, requests, among other options, that can be dynamically assigned to a user and tailored to their needs. For example, a care coach can access their console display which provides a view of the caregiver's application and the caregiver's user interface view. The coach can dynamically select, for example, homework capsules that once selected will appear in the caregiver's user interface. As part of an initial set-up or as a capsule automatically display in the user interface, a caregiver can enter information about their care recipient. FIG. 2 shows example screen captures from the mobile device user interface where a user can enter, access, or update information on their care recipient (e.g., at 202). In further embodiments, the user can access support group functionality (e.g., at 204). In various embodiments, the care coach can facilitate caregiver interactions with the mobile application and functions. For example, the care coach through their console can assign members to a support group available to a caregiver, for example, made up of other caregivers. In addition, the care coach can assign tasks that will educate a user on a caregiving task or streamline caregiving tasks, among other examples.

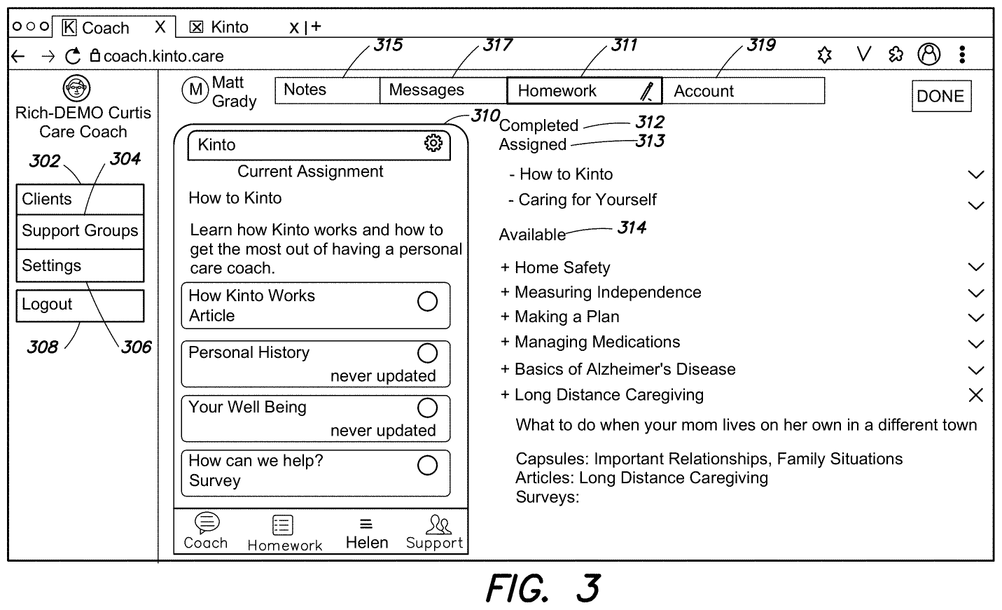

According to one embodiment, a care coach is given access to a library of capsules displayed in their app view and console (see FIG. 3 explained in greater detail below). The care coach is able to select capsules for respective caregivers, and responsive to selection trigger display in the application on the caregiver device. In one setting, a homework curriculum or library of content is based around Alzheimer's/Dementia/Cognitive Impairment conditions. In other embodiments, other libraries of capsules are tailored to different conditions, well-being regimens, treatments, etc. In some settings, capsule libraries and/or homework curriculums are tailored based on a care partner or entity (e.g., a customer, such as an employer or organization). Additionally, care coaches are given the ability to personalize the assignment set for the individual caregiver and/or libraries they access. In some examples, the personalization facilitates the relationship between coach and caregiver and helps cement the therapeutic alliance connection between them.

In some embodiments, the system delivers flexible data collection and reporting on behalf of the care recipient (e.g., often a family member or loved one). Keeping the relationship in mind, the Kinto application includes user interfaces that simplify and encourage caregivers to enter relevant data on behalf of their care recipient. This information can be used by the system to generate reports, status, well-being, and to tailor capsules and/or libraries of content for the caregiver. The reports can be used in a variety of contexts, such as informative leave-behinds for medical clinicians, substitute caregivers, or for sharing the care recipient's care information (e.g., care wishes/instructions of the care recipient) with other family members. In one example, support documentation is made readily available to caregivers to help overcome institutional hurdles faced in many care settings.

According to another embodiment, the system and/or platform can include support group interface and support functionality (e.g., FIG. 2, 204). In one example, the support interface and functionality include a chat-based support group setting in a respective mobile application (e.g., the Kinto App). In the support group interfaces, communication can be facilitated by the caregiver's coach, and include members (e.g., other caregivers, or assigned application users, etc.) of the coach's support group. In various embodiments, the communication functions and interfaces provided to the care coach enable more efficient management and streamline communication to larger audience than conventional implementation would support. In one example, a care coach is able to provide assistance to a larger support group (or sets of groups) than they would be able to otherwise manage. Through the support group functionality, the system is able to feed/surface timely concerns and themes for respective caregivers. In one example, the system leverages communication of issues, challenges, questions, etc., for a scheduled video conference support groups session. According to various embodiments, the care coach is given access to group video support session functionality through their console that enables creation and management of video support group sessions with a few clicks in the user interface (e.g., to add participants, set a schedule, a recurrence (if any), update a topic, etc.). The result of the care coach managing interactions on the caregiver's application is a holistic care solution that is dynamically configurable in real time, but that also evolves asynchronously for those times users (e.g., caregivers) are unable to directly interact with their coach. The evolving application and UI enables more efficient resource utilization and discovery. In addition, care support resources can also include automated chat-based interfaces that answer caregiver questions and improve local resource utilization and discovery. In various embodiments, the automated chat interfaces are configured to help caregivers navigate complex bureaucratic hurdles that often prevent access or efficiency in obtaining care. In some embodiments, connections between caregivers are facilitated by care coaches. In some examples, the caregivers within a care group can exchange information resources, and/or communicate their own displayed capsules with other caregivers. Various embodiments are configured to enable a permission based exchange, where the recipient can accept a communicated capsule, which can be a user interface element, or other resource before the communicated information becomes part of their application or display. In some embodiments, a care coach can manage such exchanges by controlling permissions for caregivers to exchange capsules directly, or by prohibiting exchanges generally or instance by instance. Other embodiments permit exchanges of information and/or capsules based on caregiver acceptance. In still other embodiments, the system can be configured to monitor exchanges for personal or confidential information. In one example, the system can identify a capsule template that does not include any personal information based on analyzing capsule within a requested communication. The system can then send the template for the digital object to a recipient, and the recipient does not receive any personal or confidential information. In other examples, the system can scrub personal or confidential data from a capsule before completing or triggering a communication between caregivers.

In various embodiments, the system facilitates assessment of the caregivers to determine what their individual needs are. The system is configured to tailor a caregiver mobile application to guide them. For example, a “homework” model is enabled via the care coach UI, where a care coach can introduce homework modules into the care recipient's application and display. The system provides different assignments that the care coach can select and introduce (e.g., depending on circumstances and care environment) in real time. Once committed by the care coach, the user interface at the caregiver device reflects the changed information and, for example, a new homework capsule.

In various embodiments, the display and positions of functionality groups (e.g., “Coach,” “Homework,” Care Recipient, and “Support”) are tailored to facilitate acknowledgement and to provide impetus to complete homework tasks by integrating the image of a care recipient into the user interface. In some examples, the image helps convey information, including how to manage medication, making daily plans, and scheduling activities, etc. In further embodiments, various visual modules and associated information which can include tasks and/or homework, are referred to as capsules that are displayed in the user interface for the caregiver. Capsules are implemented across the user interface and are configured to provide a universal messaging/activity format that can detail, for example, history and well-being, present surveys, deliver educational material, solicit care information, etc. In various embodiments, the care coach can introduce any capsule into the caregiver's display. During a coaching sessions, the care coach can introduce a new capsule and walk the caregiver through use of the capsule in their own display—via the App View shown to the care coach.

FIG. 3 is an example screen capture of a care coach interface. As shown, a care coach can access their clients at 302 (e.g., caregivers with respective mobile applications). The left side of the display includes options to review support groups 304 (e.g., collections of caregivers), adjust settings at 306, and logout at 308. With a client selected, the user interface is configured to show a view of a selected client's mobile application (e.g., at 310). In some examples, selection of the client can trigger display of an emulation of the client's mobile application based on a last known state or saved profile information. For example, the system can track the state of the caregiver's application (e.g., on-line, offline, inactive, etc.), and generate a connection to the caregiver device or display an emulation of the caregiver device. In FIG. 3, the care coach has navigated to current assignments in the right side of the display, which are likewise displayed on the client's device and application. In some examples, the system can support and display a live session, where action in the client device and application are reflected in the care coach interface via the view of the client application at 310, which can trigger the care coach interface to display on the right side to display relevant functions for the current application view at 310. In some examples, a mirror, tracking, or live mode allows changes in a client application to be reflected in functions displayed to the care coach, and changes in the care coach interface to affect the client application and display. In further examples, the care coach can select an option to decouple their display so that changes shown in the application view at 310 do not impact the remaining elements of the display (e.g., right side of interface). In various embodiments, the care coach interface is configured by default to permit a care coach to execute functionality at a client application via the application view, and add features or functions via the interface shown on the right side of the care coach display.

At 312, the coach interface is partitioned into sections “Completed” (e.g., at 312), “Assigned” (e.g., at 313), and “Available” (e.g., at 314). In this example, each display in the right side of the screen reflects a homework capsule that can be integrated into the client's application and display. Once completed by the client, the view updates to reflect that capsule in the respective section. As shown, “How to Kinto” and “Caring for Yourself” are displayed in the “Assigned” area. In some examples, basic capsules can be set to display to new users as part of default settings.

According to other embodiments, a series of homework capsules can be displayed for selection by the care coach, and once selected any capsule is displayed for the client (e.g., caregiver). In some embodiments, the care coach UI provides unique opportunity to tailor the application and display shown to a respective client. Additionally, the care coach UI enables the care coach to develop and manage notes (e.g., at 315), messages (e.g., at 317), homework (e.g., at 311), and account information (e.g., at 319), while interacting with a given client. In one example, care coaches leverage their expertise to adapt the user interface, homework, etc., to the client's situation as those adaptions are needed. In further example, a legal documents capsule can be integrated into the UI so the client can prepare any needed forms, and the care coach can know which documents are in place. While the platform provides more than a document repository, some legal documents become a gateway to securing care. Thus, the system can facilitate capture and communication (e.g., with your doctor or care recipient's doctor). In some settings, other caregiver users can advise on what documents may be needed for a procedure, a provider, or other circumstance on which they have experience.

According to some embodiments, each client within a care group (e.g., assigned to a care coach) can interact with each other via the support option in the UI (e.g., see FIG. 2). In that setting, clients can share with other caregivers what steps were needed (e.g., legal documents) for a specific provider or service (e.g., doctor, hospital, health plan, etc.). Thus, the group can become the beneficiary of all the accumulated knowledge and experience by employing the support functionality. In further example, a care coach can capture visual capsules from one client and copy the visual capsule to another client's mobile application, thereby facilitating information exchange, unavailable and not contemplated in many conventional implementations.

In some embodiments, the support functionality can be formatted for asynchronous sharing between members of a care group (e.g., not logged in at the same time). In other embodiments, the care group functionality can include asynchronous and synchronous functions (e.g., active login and interactive messaging). In still others, synchronous operation can be captured and preserved for asynchronous access. For example, video support sessions can be conducted on the system between a care group. The video session can be recorded and/or transcribed for access by other care recipients. In some examples, the system can integrate information from the video session into a capsule that may be selected by a care coach for integration into displays on caregiver apps who were unable to attend and/or having related issues.

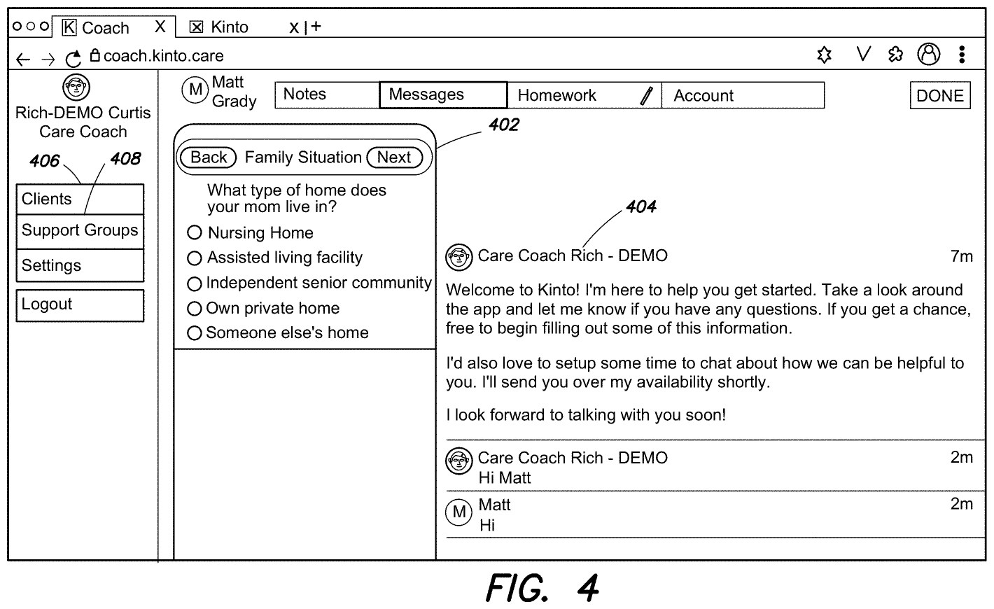

Shown at 317 is a messaging tab, responsive to selection in the UI, caused the navigation to a messaging interface (e.g., FIG. 4). As shown in FIG. 4, the message tab can be used to prompt coaches to capture information (e.g., at 402—that can later be displayed). In other examples, the messaging interface is configured to manage communication between a care coach and client (e.g., at 404), when a client is selected (e.g., 406). Alternatively, and/or in other embodiments, the message interface can be configured to manage communication with the support group (e.g., by selecting 408) and/or scheduling a support group session or meeting.

FIG. 4 illustrates an example interface for managing communication, however, other embodiments can include message capsules that can be selected by a care coach. The message capsules can include pre-formatted messages that are displayed to the client user or the support group of users responsive to selection by the care coach. In some examples, pre-formatted messages can be displayed on various topics (e.g., COVID19 recommendations, Medication instructions, preparations needed, documents needed, etc.). In some embodiments, a care coach can navigate through pre-formatted responses, optionally organized by category, select a pre-formatted response, edit and/or accept the message for communication to caregivers.

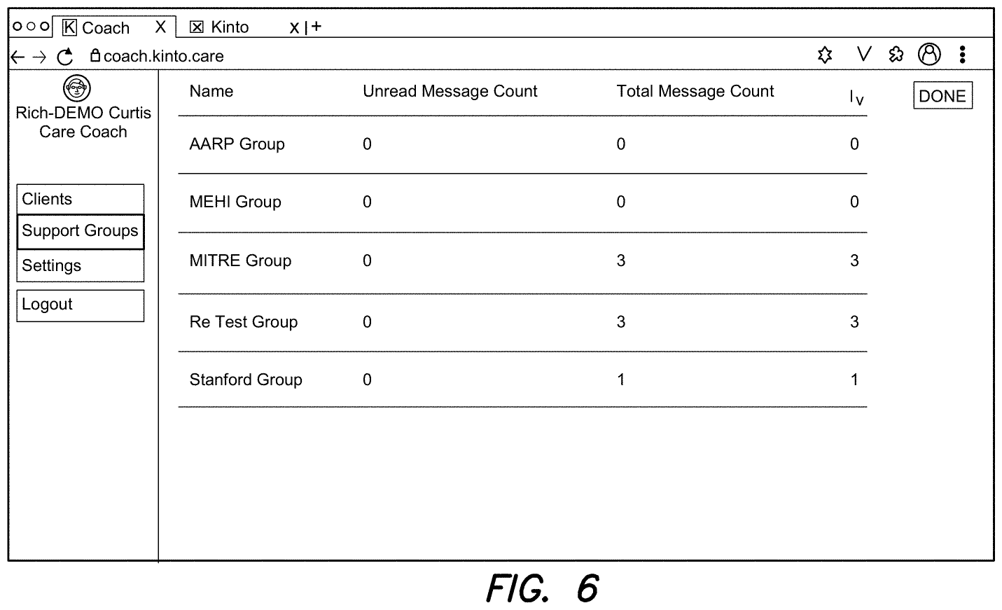

In further embodiments, a care coach can use the messaging functions to run a group support session, which can include 50 or even 100 users. As discussed above, the support session can be asynchronous, synchronous, time limited, etc. In such setting, any client and/or care coach can raise issues and rely on group knowledge for the best response. In some settings, the platform can support video-based messaging and interaction with the messaging functions and/or in a support group session. While the initial session can include interactive discussion and content sharing, the group and message exchange becomes asynchronous and available to other clients unable to attend. FIG. 6 is a screen capture of a support group interface for managing messages for any support groups under a given care coach.

According to various embodiments, the coach-controlled UI enables the system to personalize a caregiving journey for the caregiver based on, for example, an initial assessment. The system further enables updates on condition and well-being, including any circumstance that they are encountering. For example, if a care recipient experiences a fall, the care coach can update their application will fall treatment capsules as well as fall prevention capsule, that include education on environment preparation and fall risk limitations, among other options. In various embodiments, the system facilitates adjust to existing or completed capsules as well as communication of new capsules. In other embodiments, new capsules can be readily introduced via a care coach interface. For example, the care coach interface can specify and highlight new capsules for consideration.

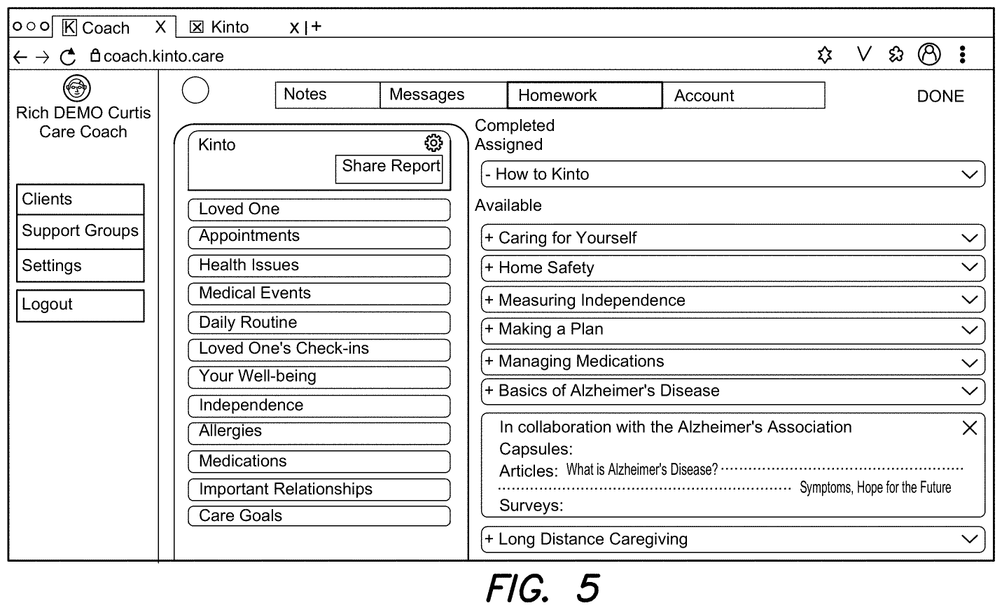

According to one embodiment, care coach interfaces and associated capsule content can be specifically tailored to a care partner. For example, various health care organizations can register with the system, and the system can identify and authenticate care coaches and/or care recipients associated with a specific health organization. Various capsules (e.g., homework capsules) can be tailored to provide entity specific instruction/education, and access to functionality specific to the health organization, for example. In one example, an instruction capsule can provide detailed information on how to ensure a health-related claim will be processed and covered by the associated health care organization. Other capsules can include information on covered (or not) services, as well as provide access to either. In further examples, care groups or care partners can include information tailored to Medicare/Medicaid operations and/or support functions, among other examples. “Insurance” capsules can collect information on a health insurance provider, as well as provide details on what information is necessary for a successful claim. In some examples, caregivers can exchange such capsules to facilitate operations for other caregivers. In other examples, care coaches can leverage visual capsules utilizes by a caregiver in their care group and introduce the same visual capsule(s) into a second caregiver's application. FIG. 5 is a screen capture of an example care coach console and associated capsules (e.g., homework capsule, among other options). The screen captures illustrate information grouping in the caregiver's user interface (e.g., loved one (e.g., care recipient), appointments, health issues, medical events, daily routine, loved one's check-ins, your well-being, independence, allergies, medications, important relationships, care goals, etc.).

In some settings, the care coach and caregivers can interact to reduce health care incidences for respective care recipients. In a long-term care setting, every day that a care recipient can stay at home instead of having moved into a Medicaid paid nursing home represents a significant saving in personnel, time, and expense, among other options. In further embodiments, other advanced care planning can be managed on the system. For example, the application, care coach, and/or care group can encourage caregivers to establish the health directives of the care recipient and store documentation of their wishes. Often final care instructions are not known or even ignored in various conventional settings. With the mobile application, a care recipient or caregiver can readily provide instructions or documentation on the care recipient's wishes for end-of-life care. Further, providing a verifiable source of such instructions ensures actions in compliance with the care recipient's wishes.

In some settings, the pairing of a care coach and caregiver can be manual, permission based, search oriented, or automated. In one embodiment, the system can include matching algorithms that looks for similarity between a care coach, the care coach's background, experience, and/or expertise, and the needs of the caregiver. Responsive to matches, one or more care coaches can be assigned to a care recipient automatically. In other examples, candidate coaches can be presented to a caregiver, and the caregiver can select a care coach from the candidates. In one alternative and/or additional embodiments, care coaches can be presented with matching caregivers, and the care coach may select one or more caregivers. In further embodiments, the system can include matching algorithms that look for similarity between a care coach, the care coach's background, experience, and/or expertise and the needs of the caregiver. In some embodiment, machine learning models can be used to identify matching care coaches and caregivers given information on a caregiver and care coach and vice versa. For example, the system can used historical data to train models on previous choices by respective caregivers of connected care coaches. By training to characteristics of the caregivers and selected care coaches the system can provide intelligent models for identifying candidate coaches. Further, the system can continually update the models with additional selections and characteristics of caregivers, among other options.

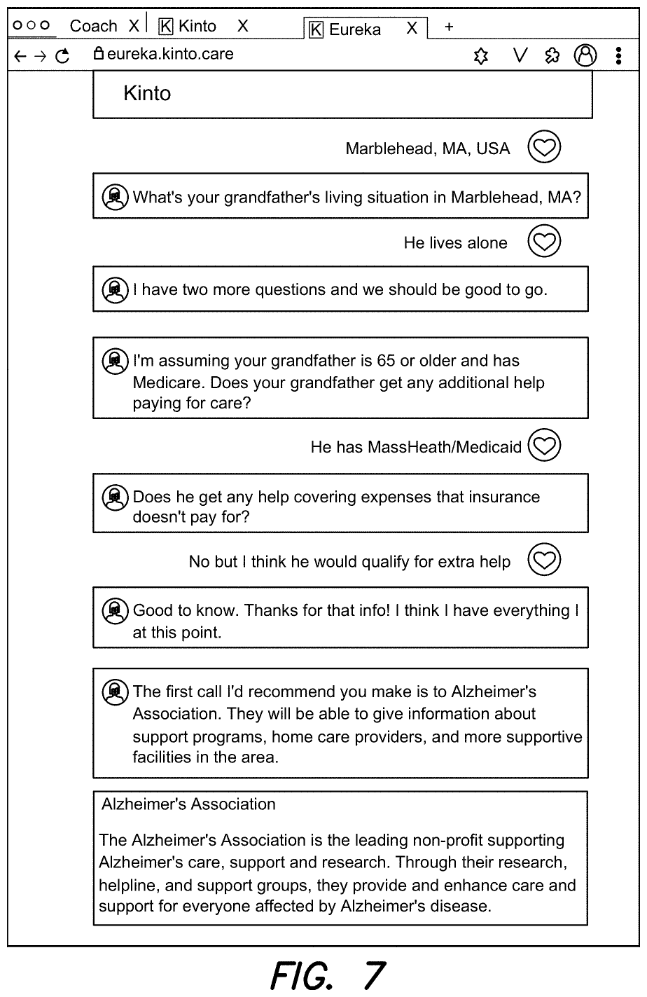

As discussed above, the care coach and caregiver pairing and integrated functionality provides a support platform that enables options unavailable in conventional implementation. In further embodiments, automated chat-based interfaces provide another opportunity for caregivers to interact with the platform. These interactions can be executed via mobile applications and/or as a web-based interface. For example, FIG. 7 illustrates a screen capture of a chat session executed with automated chat responses. The chat routines are configured to identify health issues and/or health information that can be used to deliver resource discovery information and/or provide assistance information on supporting care giving tasks. In one example, the automated chat functionality identified underlying medical conditions, living situation information, and generates a recommendation for next steps. Shown in FIG. 7, the automated chat learns about a medical condition and the care recipient's living situations. The automated chat determines that the Alzheimer's Association can provide information needed to assess, create, and/or improve a care plan or environment. According to some embodiments, chat sessions conducted via the web interface can be used by a care coach to update a caregiver's application. Even where the caregiver's application is offline, the care coach can update the caregiver's application via an emulation of the caregiver's application, where any such updates are propagated once the application is executing or available.

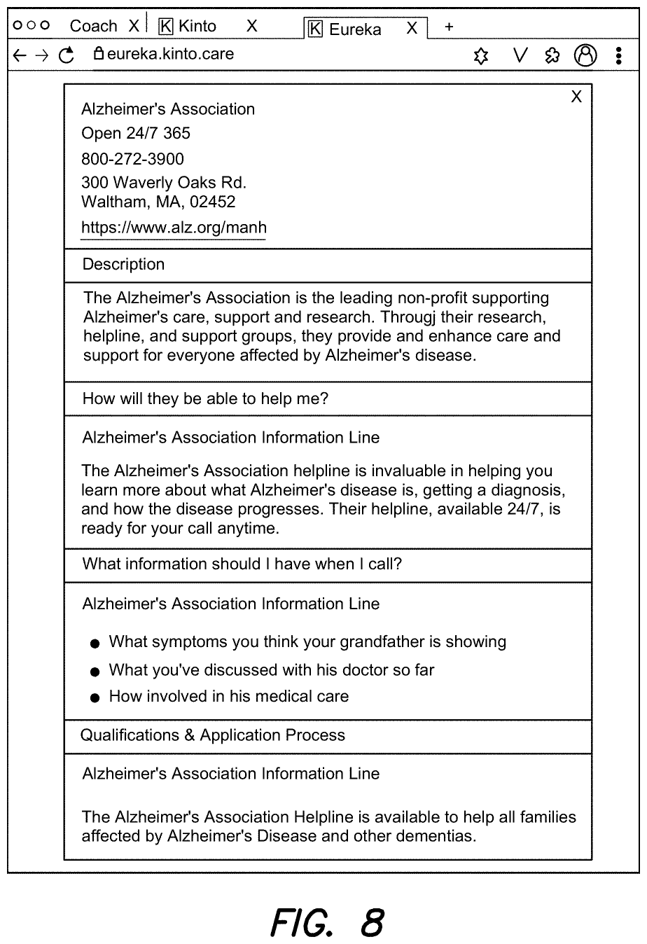

FIG. 8 illustrates a screen capture of a chat session executed with automated chat responses. As shown, the chat session describes the selected resource for the user and provides information on how the selected resource can help, how to prepare to best use the resource, and can also include information on any needed qualification and/or application process that may be required. In further example, automated session can be used to update the caregiver's application, for example, with matching capsules. The system can automatically select capsules matching concepts within an automated chat session, that will be viewable upon the caregiver's access to their application. In some embodiments, automatically identified capsules are communicated to the caregiver for acceptance prior to introduction into the caregiver's application. In other embodiments, concepts are identified in the automated chat session and communicated to a respective care coach. The care coach can then access the caregiver's application and introduce or update capsules according to the caregiver's need.

According to various embodiments, a care coach and console can be used to interact directly with linked caregivers. For example, operations executed by the care coach in the care coach console and/or application view can be mirrored in the caregiver's application, allowing the care coach to operate the caregiver's application. The care coach can teach the caregiver directly how to access or enable various functions, and/or provide needed insight. In on embodiment, the care coach console and associated computer system is configured to communicate with a central server to generate updates to respective caregiver mobile applications. The mobile applications can be configured to receive updates pushed from the central server and/or can be configured to poll the central service for respective updates. In some examples, caregiver mobile application and the care coach console can communicate directly, and updates or messages from one can be delivered directly to a caregiver application or coach console, among other options.

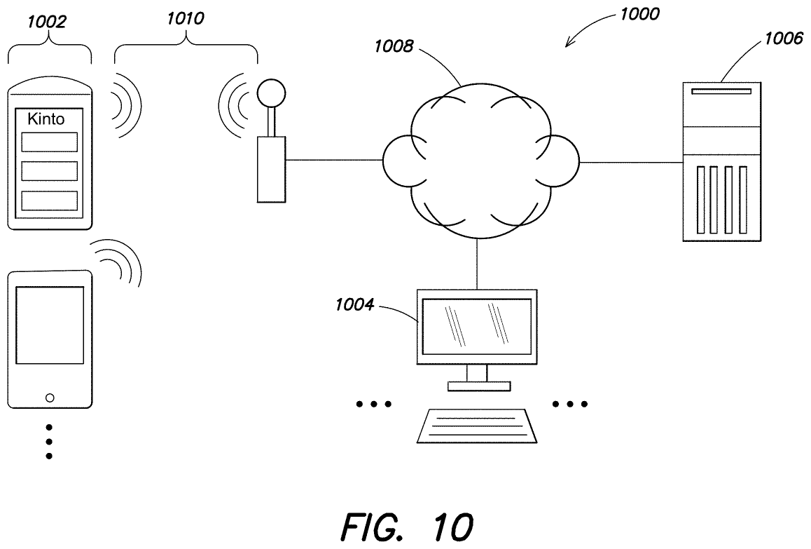

FIG. 10 shown an example system 1000 for enabling a customizable and managed user interface. According to various embodiments, the customizable and managed user interface resides on caregiver devices (e.g., 1002) and provides functionality for facilitating care environments unavailable in various conventional approaches. In some embodiments, the caregivers install a mobile application on a respective device (e.g., shown at 1002 where the three dots reflect any number of additional caregivers and devices). The mobile application includes functionality, information, and/or operations presented as visual capsules that incorporate display elements, information, etc., in the user interface.

A visual capsule can be based on a logical grouping of one or more display screens, functions, questions, information, video, educational material, etc. that are needed for respective caregiving tasks. The mobile application can be installed with a base set of visual capsules that provide starting functionality to each caregiver. In some examples, the base functionality can be tailored to the respective caregiver and their needs. In further examples, the caregiver is directed to identify their initial needs based on questions presented to the caregiver, and associated visual capsules can be presented based on the caregiver's responses. As part of the initial set up of the application, the caregiver can be paired with a care coach. In some examples, the caregiver can search for care coaches matching their needs, and in others, care coaches can select newly registered caregivers. In still other examples, the system can select and match caregivers and care coaches or present candidate coaches to caregivers and vice versa.

According to various embodiments, the inventors have realized that massive inefficiencies are introduces when care givers are required to identify, locate, access, and/or install information resources that they need without facilitators or direction. In some examples, simplifying and limiting the data presented to a caregiver to a small base set of functions improved the system (e.g., limits data required to start, limits bandwidth to communicate, etc.), and the user's ability to absorb what is presented. Additionally, linking caregivers to care coach systems that can access, control, update, and educate on functionality, makes the caregivers' mobile applications more responsive, intuitive, and constantly evolving to suit the caregivers' needs. Shown in FIG. 10 at 1004 are connected care coach system (e.g., laptops, servers, mobile devices, etc.). The caregiver devices 1002 and care coach systems 1004 (where three dots represent any number of additional systems), are configured to connect over any network (e.g., 1008 and 1010, including, for example, the Internet). In various embodiments, system 1000 can include a server 1006 that manages enrollment, authentication, etc., of caregivers and care coaches (e.g., 1002 and 1004). In various embodiments, server 1006 includes a repository or library of visual capsules that are configured to present functions, information, etc., within the mobile application. In various examples, visual capsules are user interface elements and logical groupings of display screens, functionality, questions, information sources, etc., and can be based on groupings for a caregiving task (e.g., long term fall prevention, insurance requests, end of life instructions, etc.). Each mobile application can be tailored to a care given task or needs or a care recipient by select various visual capsules related to the task or needs of the care recipient.

According to one embodiment, caregiver applications executing on caregiver devices (e.g., 1002) can be emulated on care coach systems (e.g., 1004). In this architecture, an authorized care coach can dynamically update or operate the application on the caregiver device. Updates can be executed in real time, wherein both caregivers and care coaches can update the mobile application (e.g., selecting or adding new visual capsules, updating existing visual capsules, re-ordering the user interface, adding functionality, etc.), and see the respective updates on their respective systems immediately. For example, the emulation shown to the care coach can be configured to trigger the same operations in the user interface of the application of the caregiver. In other examples, the server 1006 can maintain state information on a caregiver application/device. The emulation shown to the care coach can be based on what the application currently looks like, has installed, etc. In such examples, the care coach can update the application, user interface, functionality, etc., and such updates are stored by the server 1006, and applied to the caregiver device once available. In various embodiments, the connection between care coaches and caregivers can be asynchronous or synchronous, and permit interaction in real time, near real time, or on a delay.

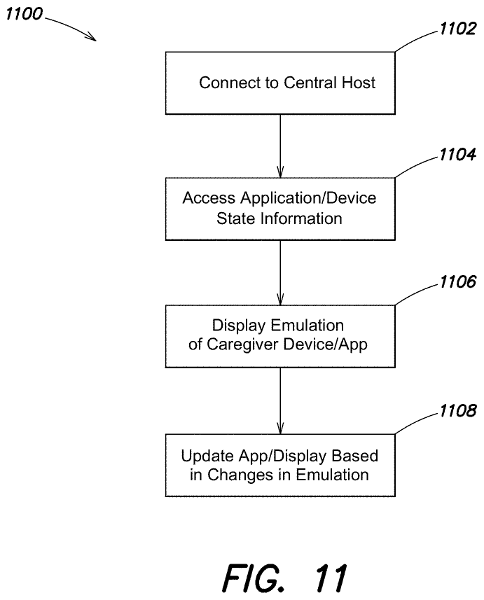

FIG. 11 is an example process flow for updating a caregiver application, according to one embodiment. Process 1100 begins at 1102 with a care coach connecting to a central host. The care coach can access a web portal, web interface, etc., and authenticate. Once authenticated, the care coach can access application/device information for their respective caregivers (e.g., coach group of caregivers and registered devices/applications) at 1104. In some examples, access to the application information can be based on stored state information or can be based on synchronous connection to the application executing on a caregiver's device, among other options. As part of the care coach accessing the application/device information, the care coach can access or display an emulation the caregiver's application or device at 1106. The care coach can update the application on the caregiver's device (e.g., introduce new visual capsules, introduce new functions, trigger operations, etc.) based on changes made in the emulation at 1108. In various examples, the emulation can reflect a synchronous connection between the caregiver device and the care coach system that operates in real time, and in others the emulation can reflect a stored state of the caregiver application, where updates are communicated to the device/application when available. Process 1100 shown various acts and/or operations as steps, in other embodiments, various ones or combinations of the steps shown can be combined or executed in different order.

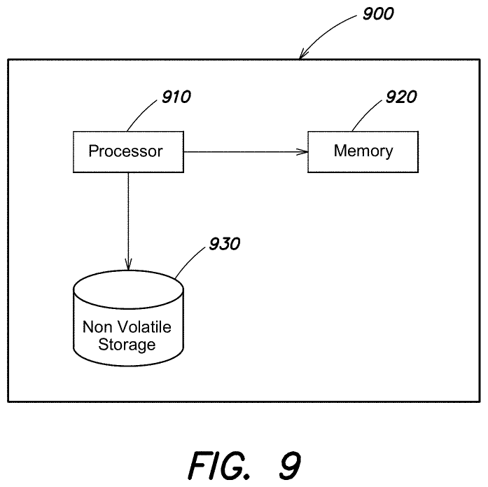

Additionally, an illustrative implementation of a special purpose computer system 900 that is improved based on the operations, functions, and/or algorithms disclose herein is shown in FIG. 9. The computer may be used and improved based on any of the embodiments of the disclosure provided herein. The computer system 900 may include one or more processors 910 and one or more articles of manufacture that comprise non-transitory computer-readable storage media (e.g., memory 920 and one or more non-volatile storage media 930). The processor 910 may control writing data to and reading data from the memory 920 and the non-volatile storage device 930 in any suitable manner. To perform any of the functionality described herein (e.g., transaction evaluation, probability analysis, re-routing transaction pathways, re-submitting transactions, etc.), the processor 910 may execute one or more processor-executable instructions stored in one or more non-transitory computer-readable storage media (e.g., the memory 920), which may serve as non-transitory computer-readable storage media storing processor-executable instructions for execution by the processor 910.

The terms “program” or “software” or “app” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the disclosure provided herein need not reside on a single computer or processor but may be distributed in a modular fashion among different computers or processors to implement various aspects of the disclosure provided herein. Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements. Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms. As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements.

This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

1. A system for enabling a customizable and managed user interface, the system comprising:
a remote computer subsystem including at least one processor operatively connected to a memory, the at least one processor configured to:
access, via a web interface, a central computer system, the central computer system for managing at least one caregiver device and at least one remote computer subsystem associated with a care coach;
access state information for an application installed on a caregiver device or access an emulation of the application executing on the caregiver device;
generate a coach console interface for the care coach on the remote computer subsystem, wherein the generation of the coach console interface includes operations to:
display an application view emulating the application executing on the caregiver device;
display a library of visual capsules, wherein at least some of the visual capsules include information on completing a care giving task and information requests to define a condition of a care recipient, and are configured to guide the caregiver through a plurality of displays to convey or capture associated information, wherein the visual capsules are associated with a respective display element; and
responsive to selection of at least one visual capsule in the coach console interface, trigger updates to the user interface for the application executing on the caregiver device to include display of the at least one visual capsule's respective display element.

2. The system of claim 1, wherein the at least one processor is configured to update the application view emulating the application executing on the caregiver device to include the selected at least one visual capsule upon execution of the at least one visual capsule executing on the caregiver device.

3. The system of claim 2, wherein the system further comprises the central computer system, and wherein the central computer system is configured to maintain state information on respective caregiver devices, wherein the state information includes application execution information including at least a list of visual capsules installed.

4. The system of claim 3, wherein the central computer system is configured to update the state information associated with respective caregiver devices responsive to changes made in the application view.

5. The system of claim 4, wherein the central computer system is configured to communicate updates to respective caregiver devices and respective applications based on the updates to the state information.

6. The system of claim 5, wherein the central computer communicates updates to respective caregiver devices in real or near real time.

7. The system of claim 6, wherein the central computer communicates updates to respective caregiver devices and the application view of the respective caregiver devices in real or near real time.

8. The system of claim 1, wherein the coach console interface includes the application view and an organization of the library of the visual capsules into sections of a user interface.

9. The system of claim 1, wherein the application view is further configured to emulate operations performed by the application executing on the caregiver device.

10. The system of claim 9, wherein operations performed within the application view are mirrored within the application executing on the caregiver device.

11. A method for managing a customizable user interface, the method comprising:
accessing, at least one processor, a central computer interface for managing at least one caregiver device and at least one management subsystem associated with a care coach;
accessing, by the at least one processor, state information for an application installed on a caregiver device or access an emulation of the application executing on the caregiver device, the state information and emulation reflecting application status and user interface settings for the application;
generating, by the at least one processor, a coach console interface for the care coach on the at least one management subsystem, wherein the act of generating includes:
displaying, by the at least one processor, an application view emulating the application executing on the caregiver device;
displaying, by the at least one processor, a library of visual capsules, wherein at least some of the visual capsules include information on completing a care giving task and information requests to define a condition of a care recipient, and are configured to guide the caregiver through a plurality of displays to convey or capture associated information, and wherein the visual capsules are associated with a respective display element; and
responsive to selection of at least one visual capsule in the coach console interface, triggering, by the at least one processor, updates to the user interface for the application executing on the caregiver device to include display of the at least one visual capsule's respective display element.

12. The method of claim 11, wherein the method further comprises updating the application view emulating the application executing on the caregiver device to include the selected at least one visual capsule upon execution of the at least one visual capsule executing on the caregiver device.

13. The method of claim 12, wherein the method further comprises maintaining, by a central computer system, state information on respective caregiver devices, wherein the state information includes application execution information including at least a list of visual capsules installed.

14. The method of claim 13, wherein the method further comprises updating the state information associated with respective caregiver devices responsive to changes made in the application view.

15. The method of claim 14, wherein the method further comprises communicating updates to respective caregiver devices and respective applications based on the updates to the state information.

16. The method of claim 15, wherein the method further comprises communicating updates to respective caregiver devices in real or near real time.

17. The method of claim 16, wherein the method further comprises communicating updates to respective caregiver devices and the application view of the respective caregiver devices in real or near real time.

18. The method of claim 11, wherein the method further comprises displaying in a coach console interface the application view and a display of the library of the visual capsules.

19. The method of claim 11, wherein the method further comprises displaying the application view to emulate operations performed by the application executing on the caregiver device.

20. The method of claim 19, wherein the method further comprises mirroring operations performed within the application view within the application executing on the caregiver device.
